(19) United States
(12) Patent Application Publication
(10) Pub. No.: US 2006/0211667 A1
(43) Pub. Date: Sep. 21, 2006

(54) Use of pregnane steroid derivatives for enhancing physical performance

(76) Inventor: Eric Marchewitz, Howell, MI (US)

Correspondence Address: Miller, Canfield, Paddock and Stone P.L.C.;c/o Robert Kelley Roth, Suite 2500, 150 West Jefferson Ave, Detroit, MI, 48226, US

(21) Appl. No.: 11/385,076

(22) Filed: Mar. 21, 2006

Related U.S. Application Data
(60) Provisional application No. 60/663,600, filed on Mar. 21, 2005.
(60) Provisional application No. 60/694,565, filed on Jun. 29, 2005.

(51) Int. Cl. A61K 31/57

(52) U.S. Cl. 514/178

(57) ABSTRACT

A method of increasing lean body mass in humans comprises administering a pregnane steroid derivative or a physiologically acceptable salt or ester thereof of the general formula: 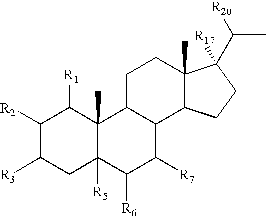 wherein R1=H and R2=H, or R1 and R2 combined form a single bond; R3 is one of OH and O; R5 is one of an α-hydrogen and a β-hydrogen; R6 is one of α-methyl and H; R7 is one of α-methyl and H; R17 is one of H and OH; and R20 is one of OH and O.

### RELATED APPLICATION

This application claims priority benefit of U.S. provisional patent applications No. 60/663,600 filed on Mar. 21, 2005 and No. 60/694,565 filed on Jun. 29, 2005.

### FIELD OF THE INVENTION

This invention relates to use of pregnane steroid derivatives for maintaining or increasing lean body mass.

### BACKGROUND OF THE INVENTION

It is desirable to maintain relatively high lean body mass (i.e., muscle mass) as a percentage of total body mass for overall good health. Benefits of maintaining lean body mass are well known, and include well-regulated metabolism, proper maintenance of body adiposity, enhanced glucose disposal and regulation of glucose levels. In addition, the maintenance of lean body mass is also associated with increased self-esteem. Maintenance of lean body mass is supported in healthy humans by many factors, including the natural production of steroid hormones such as androgens. However, as humans age the level of these naturally occurring hormones decline. The decline of these naturally occurring hormones can result in muscle wasting. Muscle wasting refers to the progressive loss of muscle mass and/or to the progressive weakening and degeneration of muscles, including the skeletal or voluntary muscles, which control movement, cardiac muscles which control the heart, smooth muscles, etc. In addition to the natural aging process there are many diseases associated with muscle wasting including some cancers, HIV/AIDS, congestive heart failure, chronic renal failure, age-related sarcopenia and anorexia/bulimia, etc. Thus, it is important for healthy individuals to maintain lean body mass as they age to achieve the aforementioned benefits.

There are several known strategies for maintaining or increasing lean body mass through the use of pharmaceuticals and dietary supplements. For example, androgens have been used extensively in the form of prohormones and/or prosteroids. The use of these androgens however, is associated with a risk of side effects including liver damage, gynecomastia, prostate enlargement, atherosclerosis, male pattern baldness, increased blood pressure and virilization to name a few. Also, the risk of abuse of androgens is very high. For these reasons, the use of androgens for the purpose of increasing lean muscle mass may be less than ideal.

Several studies have been performed looking at pregnane steroid or one of various artificially made progestagens (sometimes referred to as progestins) to increase lean body mass. These studies have reviewed the beneficial use of some high dose progestagens such as megestrol acetate and medroxyprogesterone acetate in clinical trials for the treatment of cancer related cachexia (wasting). Other research has demonstrated that pregnane steroid derivatives, in some circumstances, reduce inflammatory cytokines in skeletal muscle and also can help treat HIV related weight loss. Progesterone on its own may not, however, produce the desired level of anabolic reaction as an alternative to androgens for maintaining or increasing lean body mass. It would be desirable to provide pregnane steroid derivatives which maintain and enhance lean body mass.

### SUMMARY OF THE INVENTION

In accordance with a first aspect, a method of increasing lean body mass in humans comprises administering a pregnane steroid derivative or a physiologically acceptable salt or ester thereof. The pregnane steroid derivative can comprise, for example, 5-alpha-pregnan-6-alpha-methyl-17-ol-3,20-dione.

From the foregoing disclosure and the following more detailed description of various preferred embodiments it will be apparent to those skilled in the art that the present invention provides a significant advance in the methods for maintaining and increasing muscle mass. Additional features and advantages of various preferred embodiments will be better understood in view of the detailed description provided below.

### DETAILED DESCRIPTION OF CERTAIN PREFERRED EMBODIMENTS

It will be apparent to those skilled in the art, that is, to those who have knowledge or experience in this area of technology that many variations are possible for the method of maintaining or increasing lean body tissue, such as muscle mass, disclosed here. The following detailed discussion of various alternative and preferred features and embodiments will illustrate the general principles of the invention with reference to improved method of maintaining or increasing muscle mass through the use of orally available dietary supplements. Other embodiments suitable for other applications will be apparent to those skilled in the art given the benefit of this disclosure.

Pregnane steroid derivatives can be used by the body instead of androgen in the pathways associated with maintaining and enhancing lean muscle mass. The present invention discloses a method of administering a pregnane steroid derivative, 5-alpha-pregnan-6-alpha-methyl-17-ol-3,20-dione, or other similar pregnane steroid derivatives, or physiologically acceptable salt or ester thereof for maintaining or increasing lean body mass in mammals such as humans without the side effects associated with the use of androgenic hormones. Progesterone increases lean body mass through several mechanisms including the reduction of inflammatory cytokines in skeletal muscle. Also, 5-alpha reduction of pregnane steroid derivatives has been shown to play a role in the modulation of GABA receptor function. 5-alpha reduction of the disclosed pregnane steroid derivatives increases androgen receptor binding affinity when compared with progesterone.

As used herein, a derivative of a compound refers to a species having a chemical structure that is similar to the compound, yet containing a chemical group not present in the compound and/or deficient of a chemical group that is present in the compound. The substance to which the derivative is compared is known as the “parent” substance. Here, for example, the parent compound is pregnane steroid, or 4-pregnene-3,20-dione. A derivative may be made by modification of the parent compound or by synthesis from other starting materials that are not similar to the parent.

Preferably the pregnane steroid derivative is of the general formula: 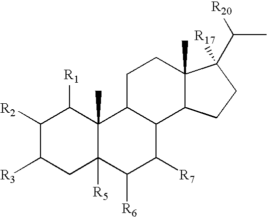
wherein R1=H and R2=H such that the resulting compounds formed are pregnans, or R1 and R2 combined form a single bond so that the resulting compound is a 1-pregnen; R3 is one of OH and O; R5 is one of an α-hydrogen and a β-hydrogen, where alpha or α refers to a functional group below the plane of the molecule and beta or β refers to a functional group above the plane of the molecule; R6 is one of α-methyl and H; R7 is one of α-methyl and H; R17 is one of H and OH; and R20 is one of OH and O. When R3 and/or R20 are OH, it is understood that the fourth bond to the carbon is hydrogen. Pregnane steroid derivatives made according to the above formula can comprise, for example: 5-alpha-pregnan-6-alpha-methyl-17-ol-3,20-dione; 1-pregnen-7-alpha-methyl-17-ol-3,20-dione; 1-pregnen-6-alpha-methyl-17-ol-3,20-dione; 5-beta-pregnan-6-alpha-methyl-17-ol-3,20-dione; 5-alpha-pregnan-7-alpha-methyl-17-ol-3,20-dione; 5-beta-pregnan-7-alpha-methyl-17-ol-3,20-dione; 1-pregnen-7-alpha-methyl-3,20-dione; 1-pregnen-6-alpha-methyl-3,20-dione; 5-beta-pregnan-6-alpha-methyl-3,20-dione; 5-alpha-pregnan-7-alpha-methyl-3,20-dione; 5-beta-pregnan-7-alpha-methyl-3,20-dione; 5-alpha-pregnan-6-alpha-methyl-3,20-dione; 5-alpha-pregnan-17-ol-3,20-dione; 5-beta-pregnan-17-ol-3,20-dione; 1-pregnen-17-ol-3,20-dione; 5-alpha-pregnan-6-alpha-methyl-3,17-diol-20-one; 5-beta-pregnan-6-alpha-methyl-3,17-diol-20-one; 5-alpha-pregnan-6-alpha-methyl-3,17,20-triol; 5-beta-pregnan-6-alpha-methyl-3,17,20-triol; 5-alpha-pregnan-7-alpha-methyl-3,17-diol-20-one; 5-beta-pregnan-7-alpha-methyl-3,17-diol-20-one; 5-alpha-pregnan-7-alpha-methyl-3,17,20-triol; 5-beta-pregnan-7-alpha-methyl-3,17,20-triol; 1-pregnen-7-alpha-methyl-3,17-diol-20-one; 1-pregnen-7-alpha-methyl-3,17,20-triol; 1-pregnen-6-alpha-methyl-3,17-ol-20-one; 1-pregnen-6-alpha-methyl-3,17,20-triol; 1-pregnen-6-alpha-methyl-3-ol,20-one; 1-pregnen-6-alpha-methyl-3,20-diol; 1-pregnen-7-alpha-methyl-3,20-diol; 5-alpha-pregnan-17-ol-3,20-dione; 5-alpha-pregnan-3,17-diol-20-one; 5-alpha-pregnan-3,17,20-triol; 5-beta-pregnan-17-ol-3,20-dione; 5-beta-pregnan-3,17-diol-20-one; 5-beta-pregnan-3,17,20-triol; 1-pregnen-3,17-diol-20-one; and 1-pregnen-3,17,20-triol, as well as physiologically acceptable salt or esters thereof. Other pregnane steroid derivatives suitable for maintaining or increasing lean muscle mass will be readily apparent to those skilled in the art, given the benefit of this disclosure.

All of the naturally occurring pregnane steroid derivative compounds disclosed herein, including, 5-alpha-pregnan-6-alpha-methyl-17-ol-3,20-dione, would preferably be administered orally mixed with solid or liquid carriers in appropriate unit doses for the purpose of increasing lean body mass.

The preferred amount of the active ingredient that is to be administered, such as orally would depends on various factors such as the age and weight of the user. An effective oral daily dosage of the described derivatives including 5-alpha-pregnan-6-alpha-methyl-17-ol-3,20-dione might be 25 to 1000 mg. In one embodiment the preferred range can be 100 to 300 mg per day. A preferred embodiment might be to administer the oral dose as a soft gelatin capsule or oral liquid suspension, either in two to three divided doses per day (i.e., 50 to 150 mg twice per day, or 35 to 100 mg three times per day). Pregnane steroid derivatives may also be administered transdermally using acceptable liquid vehicles, sublingually, transrectally (by suppository) intranasally, intravenously, subcutaneously, or by intramuscular injection.

Pregnane steroid derivatives as disclosed herein may also be mixed with weight reducing pharmaceuticals or dietary supplements such as caffeine, creatine, synephrine, or ephedrine if desired. For example, a gel capsule could comprise 50-100 mg of a pregnane steroid derivative and 400-450 mg of creatine.

### EXAMPLE 1

Capsules. 7.5 kg of the pregnane steroid derivative, 5-alpha-pregnan-6-alpha-methyl-17-ol-3,20-dione are mixed with 2.5 kg maltodextrin, and placed into 100,000 hard-gelatin capsules. Each capsule contains 75 mg of 5-alpha-pregnan-6-alpha-methyl-17-ol-3,20-dione.

### EXAMPLE 2

Capsules. 1.5 kg of 5-alpha-pregnan-6-alpha-methyl-17-ol-3,20-dione is mixed with 8.5 kg creatine, and placed into 20,000 hard-gelatin capsules. Each capsule contains 75 mg of 5-alpha-pregnan-6-alpha-methyl-17-ol-3,20-dione and 425 mg creatine. Creatine is mixed in to aid in muscle development.

From the foregoing disclosure and detailed description of certain preferred embodiments, it will be apparent that various modifications, additions and other alternative embodiments are possible without departing from the true scope and spirit of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A method of increasing lean body mass in humans comprising administering a pregnane steroid derivative or a physiologically acceptable salt or ester thereof of the general formula: 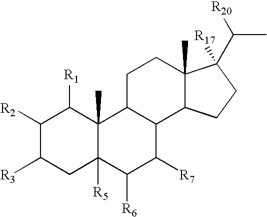
wherein
R1=H and R2=H, or R1 and R2 combined form a single bond;
R3 is one of OH and O;
R5 is one of an α-hydrogen and a β-hydrogen;
R6 is one of α-methyl and H;
R7 is one of α-methyl and H;
R17 is one of H and OH; and
R20 is one of OH and O.

2. The method of claim 1 wherein the pregnane steroid derivative is administered in one of the following ways: orally, transdermally, intranasally, by injection, sublingually, and transrectally.

3. The method of claim 1 wherein the pregnane steroid derivative is administered as a daily dosage between about 25 mg and 1000 mg.

4. The method of claim 3 wherein the pregnane steroid derivative is administered as a daily dosage between about 25 mg and 1000 mg.

5. The method of claim 4 wherein the pregnane steroid derivative is administered as a daily dosage between about 100 mg and 300 mg.

6. The method of claim 5 wherein the pregnane steroid derivative is administered as a daily dosage between about 50 mg to 150 mg twice per day.

7. The method of claim 5 wherein the pregnane steroid derivative is administered as a daily dosage between about 35 mg to 100 mg three times per day.

8. The method of claim 1 wherein the pregnane steroid derivative is administered in the form of a gel capsule.

9. The method of claim 1 further comprising administering about 50-100 mg of the pregnane steroid derivative and about 400-450 mg creatine.

10. The method of claim 1 wherein the pregnane steroid derivative is administered in the form of a liquid suspension.

11. The method of claim 1 wherein R3 is O, R17 is OH and R20 is O.

12. The method of claim 1 wherein R5 is α-Hydrogen.

13. The method of claim 1 wherein R6 is α-methyl.

14. The method of claim 1 wherein R1 and R2 are H.

15. The method of claim 1 wherein R1 and R2 combined form a single bond.

16. The method of claim 1 wherein R5 is β-hydrogen.

17. The method of claim 1 wherein R7 is α-methyl.

18. The method of claim 11 wherein R5 is α-Hydrogen, and R6 is α-methyl.
